(19) United States
(12) Patent Application Publication
(10) Pub. No.: US 2011/0288560 A1
(43) Pub. Date: Nov. 24, 2011

(54) SYSTEM AND METHOD FOR TELESURGERY

(76) Inventors: Shaul Shohat, Kfar Haoranim (IL); Eli Machlev, Shaar Efraim (IL); Roni Shabat, Kibutz Izrael (IL)

(21) Appl. No.: 12/309,769

(22) PCT Filed: Jul. 29, 2007
(86) PCT No.: PCT/IL2007/000943

§ 371 Date: Jan. 29, 2009

Related U.S. Application Data
(60) Provisional application No. 60/834,564, filed on Aug. 1, 2006.

(51) Int. Cl. A61B 19/00

(52) U.S. Cl. 606/130

(57) ABSTRACT

A system for telesurgery having a plurality of operative end units respectively linked to remote master end units. All the master and operative end units of the system consist of a common module having a base plate to which a slender shaft is slidingly attached. A slender shaft is further rotatable about its axis as well as about a center point disposed above the distal surface of the base plate. Surgical effectors, cameras and/or LEDs can be attached to the distal end of slender shafts of operative end units which is distally disposed relative to the base plate of its respective operative end unit. Operating handles for manipulating and/or activating the respective slender shaft or attached surgical effectors, are attached to the proximal end and/or to the distal end of slender shafts of master end units. Linking a master end unit to its respective operative end unit is accomplished by mechanical, hydraulic, pneumatic and or electric transmission, such that a slender shaft of an operative end units simultaneously move in the same movement in which the slender shaft of the respective master end unit is moved. An operator is able to manipulate a slender shaft within the lumen enclosing the surgical site within a patient's body by sliding, inclining and/or rotating the slender shaft of a remote master end unit as in a common endoscopic procedure. The operator is further able to manipulate and/or activate a surgical effector either as in an endoscopic or in an open surgery by means of the operating handles of a remote master end unit.

### FIELD OF THE INVENTION

The present invention relates in general to telesurgery and minimal invasive surgical operations. More particularly the present invention relates to surgical systems having a plurality of operative end units respectively coupled to remote master units, surgical effectors, cameras and LEDs attached to respective end units for in situ operating, illuminating, displaying and/or imaging.

### BACKGROUND OF THE INVENTION

Surgical procedures carried out at a distance thanks to advanced robotic and computer technologies are known. Systems for telesyrgery are common in the marketplace. One of the early demonstrations of trans-Atlantic telesurgery was reported in 2001 when surgeons in New York operated on a patient in Strasbourg, France and used remote-controlled robots to resect the gall bladder by laparoscopy.

Quite often endoscopic procedures impose on the physician tedious postures that may cause tremor and reduce the accuracy and efficacy of the procedure. Hence systems providing for telesurgery could be beneficial in this respect. However common robotic systems for telesurgery require quite sophisticated infrastructure and human resources that are normally hard to get. Therefore any system for telesurgery that is simple to manufacture and to operate is beneficial.

### BRIEF DESCRIPTION OF THE DRAWINGS

FIG. 1 is a simplified block diagram of a system for telesurgery of the invention;

FIG. 2A is a schematic presentation of a common module of a system for telesurgery of the invention;

FIG. 2B is a schematic presentation of an operative end unit of a system for telesergery of the invention;

FIG. 2C is a fragmented isometric view of a segment of a common module of a system for telesurgery according to a preferred embodiment of the present invention;

FIG. 3 is a scheme describing an exemplary mechanical linking of translational motion of the slender shafts of two coupled common modules of a system for telesurgery of the present invention;

FIG. 4A is an elevational view of a slender shaft having a surgical effector attached to its distal end in accordance with an embodiment of the present invention;

FIG. 4B is an elevational view of a slender shaft having a surgical effector attached to its distal end in accordance with a preferred embodiment of the present invention;

FIGS. 5A-5C are isometric and elevational views of an exemplary introducer of a system for telesurgery of the invention respectively;

FIG. 5D is an isometric view of the distal segment of an introducer of a system for telesurgery according to a preferred embodiment of the present invention;

FIG. 6A-6B are two elevational views of a surgical effector attached to a mounting frame of a system for telesurgery in accordance with a preferred embodiment of the present invention;

FIGS. 7A-7B are elevational views of linking accessories of a system for telesurgery in accordance with three different embodiments of the present invention respectively;

FIGS. 8A-8B are schematic presentations of two slender shafts according to two different embodiments of the present invention respectively;

FIG. 9A is a schematic presentation of a remote viewing member of a viewing subsystem of a system for telesurgery in accordance with a preferred embodiment of the present invention attached to a patient's body;

FIGS. 9B-9C are schematic presentations of a control member of the viewing subsystem shown in FIG. 9A according to two preferred embodiments of the present invention;

FIG. 10 is a schematic presentation of a master unit of a system for telesurgery according to a preferred embodiment of the present invention;

### DETAILED DESCRIPTION OF THE PRESENT INVENTION

In accordance with the present invention a system and method for telesurgery is provided. The system of the invention has one or more master end units respectively linked to operative end units. A system of the invention further has various accessories such as surgical effectors, introducers, viewing and/or imaging devices and linking devices as is further described infra. All of the end units, the master end units and the operative end units comprise a common module having a rotatable slender shaft slidingly attached to a base plate. Handles and/or operating levers, such as those providing for manually activating and manipulating common endoscopic surgical devices, are mounted onto the slender shaft of a master end unit. By manipulating the slender shaft of a master end unit and/or its associated operating handles, the surgeon causes the slender shaft of the respectively coupled operative end unit to move, such that every movement of the shaft of the master unit is identically tracked and followed. Similarly surgical effectors or devices attached to the distal end of a shaft of an operative end unit are activated by dedicated handles of the respective master unit. Manipulating the handles of the master unit is effected in the same manner as is normally applied whilst operating common surgical equipment during endoscopic and/or open surgery procedures, as is further described infra.

System for Telesurgery

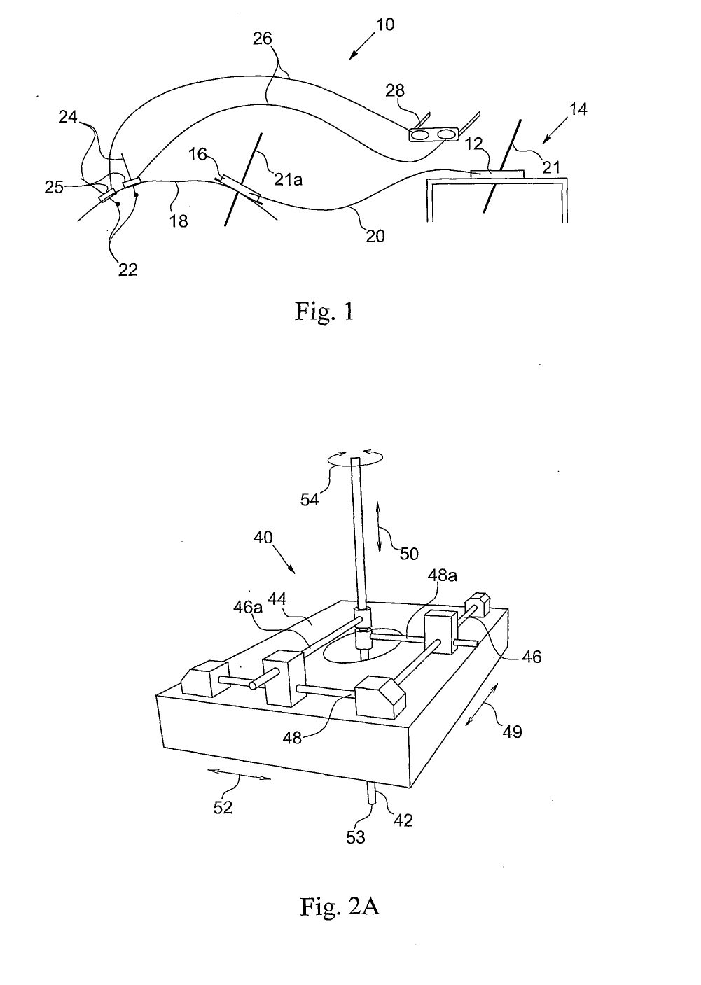

Reference is now made to FIGS. 1-3. In FIG. 1 system for telesurgery 10 according to the preset invention is schematically shown. One or more master end units such as master end unit 12 are mounted onto remote work station 14. Operative end units, such as end unit 16, are disposed on surface 18 of a patient's body adjacent to a surgical aperture cut through its wall close to the surgical site. Links such as link 20, provide for coupling between the movements of slender shafts 21 and 21a such that shaft 21a identically moves while tracking each and every movement of shaft 21. Moving slender shaft 21 is manually effected by the surgeon. Coupling between the respective shafts can according to the present invention be mechanically, pneumatically, hydraulically and/or electrically accomplished. Link 20 is implemented according to a preferred embodiment of the present invention by hydraulic actuators attached to the respective slender shafts connected by suitable piping. Similarly stretched cables and pulleys provide for such coupling according to another preferred embodiment as is further described infra. Miniature cameras 22 such as consisting of charge coupled devices (CCD) and associated optics, light sources, such as light emitting diodes (LEDs), not shown, respectively attached to the distal ends of slender shafts 24 of operative end units 25 provide for stereoscopic viewing and/or in situ imaging. The location and pointing of each camera is similarly controlled according to the present invention by means of dedicated master end units, not shown, respectively mounted onto workstation 14. Fiber optic cables 26 provide according to a preferred embodiment of the present invention for feeding each eyepiece of binoculars 28 with its corresponding image as reflected by the respective camera. Optionally, electrical either wired, or wireless, link substitutes the fiber-optic link, such that the stereoscopic features of the image are retained. A pair of operative end units respectively equipped with cameras and their coupled master units and the associated viewing device are regarded hereinafter as a viewing subsystem of a system for telesurgery of the invention, to be further described infra.

The components of a system for telesurgery of the invention are made of common materials typically utilized in manufacturing tools and equipment for endoscopic procedures and/or open surgery operations.

Common Module

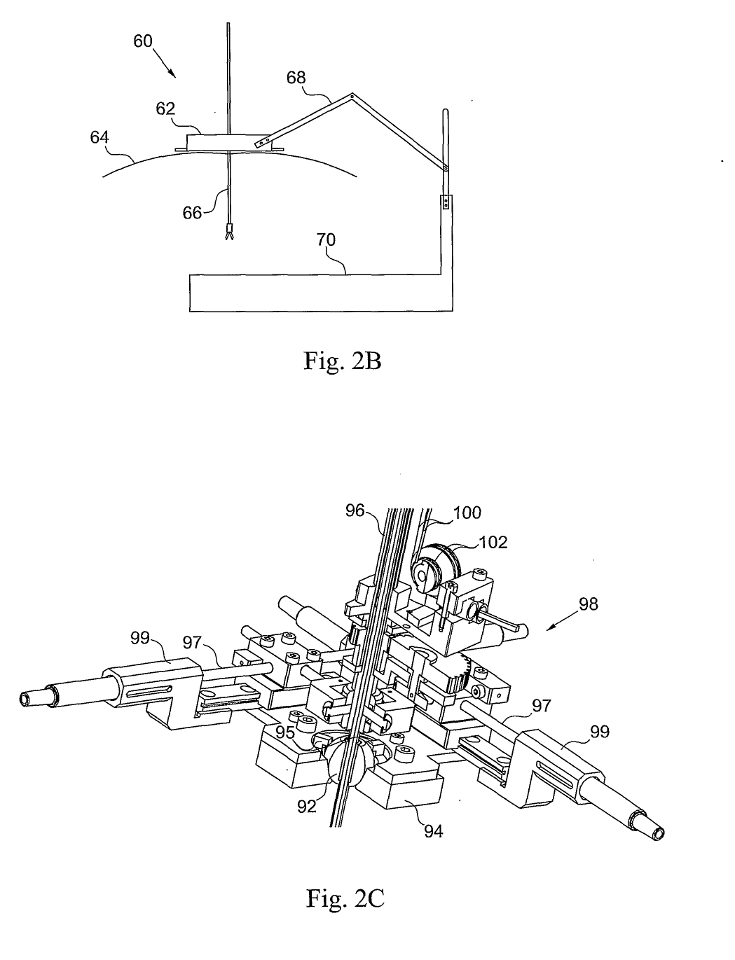

In FIG. 2A common module 40 of a system for telesurgery of the invention is schematically shown. Common modules of all end units are identical except for some slight modifications and/or additions, such as operative handles or levers attached to a common module of a master end unit, or a connecting brackets and/or mounting frames attached to the distal end of a slender shaft for connecting a surgical effector, such as scissors or CCD camera, thereto. Slender shaft 42 is slidingly attached to base plate 44 by means of a sliding bearing, not shown. Rods 46 and 48 are firmly attached to base plate 44. Rod 46a is slidingly attached to rod 48 and to slender shaft 42. For the sake of simplicity for better describing the features of the invention a Cartesian coordinate system is introduced. The axes of the coordinate system are as follows: the x-axis is parallel to rod 46a, the y-axis is parallel to rod 48a and the z-axis lies along the slender shaft when is perpendicular to base plate 42. The origin of the coordinate system is disposed at the center of the sliding bearing, not shown, connecting slender shaft to base plate 44. Slender shaft 42 is rotatable about any radial axis contained in the x-y plane and whose azimuth angle is any angle in the range [0,2π]. Slender shaft 42 is further rotatable by an elevation angle its magnitude cannot exceed a predefined value. Any rotation of slender shaft 42 relative to the y-axis results in a movement of rod 46a in a respective direction of double arrow 49 simultaneously with a slight movement of its end along slender shaft 42, while rod 48a stays fixed in place. Similarly any rotation of slender shaft 42 relative to the x-axis induces a respective translational motion of rod 48a simultaneously with a slight movement of its end along slender shaft 42, wile rod 46a stays fixed in its original place. Any rotation of the slender shaft by an elevation angle θ relative to an axis in the x-y plane whose azimuth is φ is equivalent to two successive rotations one of which is a rotation of the slender shaft by the elevation angle θ relative to the y-axis and the other is a rotation of the inclined slender shaft by the angle φ relative to the z-axis. Therefore a rotation of the slender shaft relative to an axis within the x-y plane having any azimuthal angle is equivalent to a pair of lengths Δx, Δy along which rods 46a and 48a respectively translate. Furthermore, slender shaft 42 is slidable relative to base plate 44; its distal end 53 can be lowered down to, and/or raised up to, any desired level by a distance Δz below base plate 44. Such sliding is accomplished by respectively pushing slender shaft towards, or pulling it off, base plate 44. Slender shaft 42 is further rotatable relative to its longitudinal axis by any angle in the range of [0,2π] in any of the directions shown by double arrow 54.

In FIG. 2B common module 70 of an operative end unit of the invention is shown disposed on a surface of a patient's body. The distal face of base plate 62 is coated with a suitable adhesive, as known, providing for attaching it to surface 64 of the patient's body. Such attaching provides for sealing off the surgical aperture cut through the wall of the patient's body through which slender shaft 66 is introduced. Common module 60 of this operative end unit is secured in place by articulated arm 68 firmly connecting it to a rod extending off a side of operating bench 70.

In FIG. 2C a fragmented isometric view of a segment of common module 90 according to a preferred embodiment of the present invention is shown. Sliding bearing 92 is spherical. It is disposed in an elliptic recess having two apertures respectively disposed on the proximal and the distal surfaces of base plate 94. A bore whose radius is fitted to the radius of slender shaft 96 is drilled through sliding bearing 92. Optionally the inner surface of the bore is coated with a film, such as film made of Teflon, interleaving between slender shaft 96 and the surface of the bore. A number of brackets, such as brackets 95, which are optionally made of Teflon, press against the spherical surface of sliding bearing 92 providing for its rotation about its center, while keeping the center fixed in its place within the recess. The point relative to which a slender shaft is rotatable such as the center point of the aforementioned sliding bearing is referred hereinafter as the center point. Rods 97 are firmly attached to top member 98 which is slidingly attached to base plate 94 by means of sliding bearings 99. Sliding bearing 92 simultaneously with slender shaft 96 are rotated by inclining slender shaft to a desired direction [φ,θ]. Such rotations bring about the translations of rods 97 by Δx and Δy respectively, as described hereinabove. A pair of stretched cables 100 and pulleys 102 provide for inducing translational motion of the slender shaft of an operative end unit coupled to its respective master unit by the above mentioned distance Δz.

Linking a Master Unit to its Coupled Operative End Unit

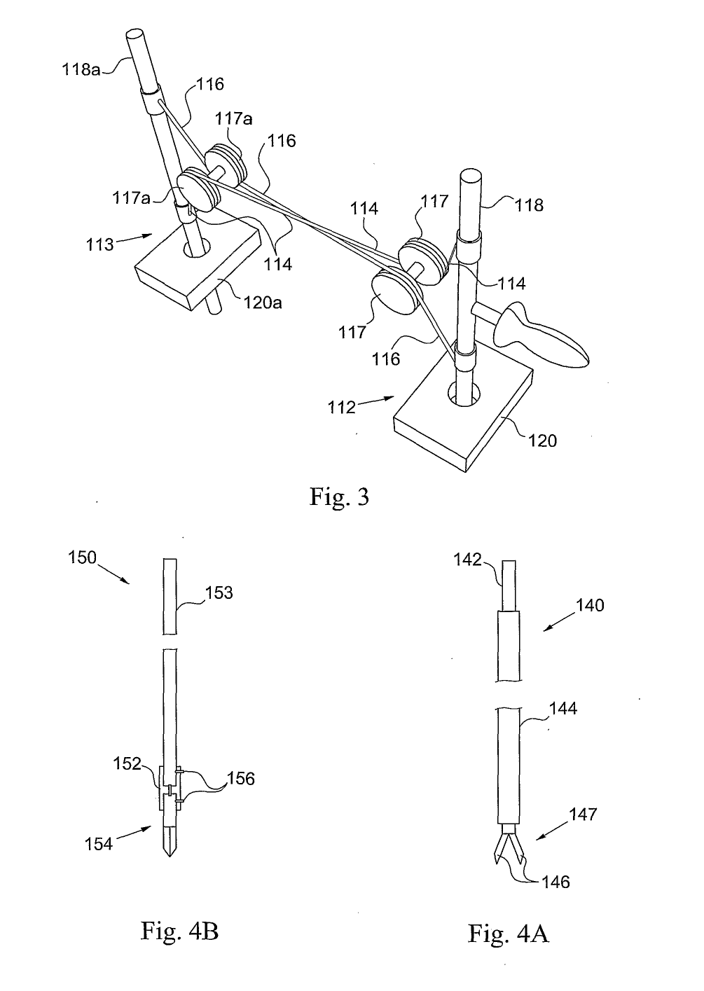

An exemplary scheme describing mechanical transmission system linking between master end unit 112 and its respectively coupled operative end unit 113 is shown in FIG. 3. Both end units 112 and 113 have the same common member described above with reference to FIG. 2C. Cables 114 and 116 provide for mechanically linking the simultaneous translational motion along a distance Δz of the slender shafts of both end units. One end of stretched cable 114 is connected to a point close to the proximal end of the slender shaft of master end unit 112. The other end of cable 114 is connected to a point distally located on the slender shaft of operative end unit 113, which is proximally disposed relative to upper member 120a. On the other hand, one end of cable 116 is connected to a point disposed close to the proximal end of the slender shaft of operative end unit 113 whereas the other end of cable 116 is connected to a point distally located on the slender shaft of master end unit 112 which is proximally located relative to upper member unit 120. Pulleys 117 and 117a are fixed relative to upper members 120 and 120a respectively. The distance between both connecting points along both slender shafts is the same and it fits the maximal distance to which the distal end of the slender shaft of operative end unit 113 can extend into the lumen of a body of a patient enclosing the surgical site.

The translational motion of the top members of two coupled common modules along one axis, say the x-axis, can be similarly linked by mechanical transmission system consisting of a pair of stretched cables and two pairs of pulleys, the pulleys of each of which are engaged with a respective cable. Alternatively, an hydraulic transmission system may according to the invention implement such link. Namely, each end of any of the rods 46a and 48a of the top member described above with reference to FIG. 2C to which reference is again made is connected to a respective hydraulic actuator. In such a case, a pair of hydraulic actuators connected to the ends of two corresponding rods one of which belongs to a master end unit and the other to its respectively coupled operative end unit, are mutually connected by a hydraulic pipe filled with a biocompatible fluid, such as water. The pipe connects actuators respectively attached to two opposing ends of the respective rods, such that pushing an end of one rod corresponds to pulling the opposite end of the respective rod of the other common module, such that both rods move along the same distance and in the same direction. Such hydraulic transmission system provide for transmitting the translations of the top member of a master end unit to respectively move the top member of its coupled operative end unit. Such linking provide for inducing identical and simultaneous motion of both top members. Such simultaneous motion is accomplished by rotating the slender shaft of the respective master end unit and thereby moving the top members of both end units by the same distances Δx, Δy. Linking can also be according to the invention electrically applied, such as by means of linear actuators or control DC motors associated with suitable electrical logic as known. Similarly the rotational motion of the slender shafts around their longitudinal axes can be linked according to the invention by any of the above mentioned techniques. Operative handles attached to slender shaft of a master end unit for activating and/or manipulating a surgical effector attached the slender shaft of the respectively coupled operative end unit are similarly linked by any of the transmissions systems described above, as is further described infra.

In each of the cases described above the transmission of the rotational and/or translational motions retains a one to one correspondence between the magnitudes and directions of the motion associated with the slender shaft of a master end unit and the tracking motion of its respective follower. Namely translations and or rotations of the slender shaft of an operative end unit are identically the same as induced by the operator inclining, rotating and/or sliding the slender shaft of the respectively coupled master unit. Optionally mechanical, hydraulic, pneumatic and or electric gear transmissions can be incorporated into any link between any pair of coupled end units thereby the magnitude of the transmitted motion is amplified or attenuated as desired. However preferable are transmission ratios of one to one as in such cases the operator performs as if manipulating directly the end units in situ. Therefore amplification of the transmitted motion should compensate only for losses along a link if any such losses exist, such that the haptic feedback to the surgeon is the same as if while he or she is manipulating the slender shaft of the respective operative end unit.

A surgeon is able to place the distal end of the slender shaft of an operative end unit at a desired location having distinct x, y, z coordinates relative to a fixed coordinate system within the lumen of a patient's body by respectively inclining, rotating and/or sliding the slender shaft of the respectively coupled master unit. The surgeon is further able to rotate the slender shaft of an operative end unit such that an effector attached to its distal end points in a desired direction, by similarly rotating the slender shaft of the respective master unit by the suitable angle to the same direction. The surgeon is further able to activate and manipulate a surgical effector as a whole and/or members of a surgical effector attached to a slender shaft of an operative end unit, by manipulating operating handles attached to the slender shaft of the respectively coupled master unit.

Accessories and Slender Shafts

Reference is now made to FIGS. 4A-6B. Slender shaft 140 shown in FIG. 4A, consists of inner shaft 142 enclosed within external sheath 144. Surgical effector 146, such as scissors, is disposed at the distal end of slender shaft 140. Biasing spring, not shown, internally disposed within sheath 144 provides for either opening, or closing, arms 146 when is released. Opening wide both arms and further releasing the biasing spring to close them, or alternatively releasing the biasing spring to open both arms and further closing them are accomplished by means of an opening/closing mechanism installed within the proximal end of sheath 144, not shown. The opening and further closing can be accomplished either by repeatedly pressing inwards the proximal end of inner shaft 142 into sheath 144, or by a reciprocating movement inwards and outwards of the inner shaft 142 into, or off, sheath 144. Slender shaft 140 can be introduced through the respective sliding bearing of an operative end unit of the invention into a lumen enclosing the surgical site within a patient's body.

In FIG. 4B a slender shaft of a system for telesurgery according to a preferred embodiment of the present invention is shown. Slender shaft 150 is tubular providing for introducing an inner shaft or cables for operating various surgical effectors attached to its distal end. Mounting frame 152, which is releasably attached to the distal end of pipe 153, provides for releasably attaching various surgical effectors, such as effector 154. Locking devices 156 provide for attaching either the mounting frame to pipe 153, or effector 154 to frame 152. An exemplary snapping mechanism having a pair of levers resiliently attached to mounting frame 152 whose respective ends are sprung into recesses respectively disposed on the sidewall of pipe 153 and the surface of the body of effector 154 serve as locking devices according to a preferred embodiment of the present invention. For releasing effector 154 off frame 152, or releasing frame 152 off pipe 153, the respective lever is laterally pulled off its recess.

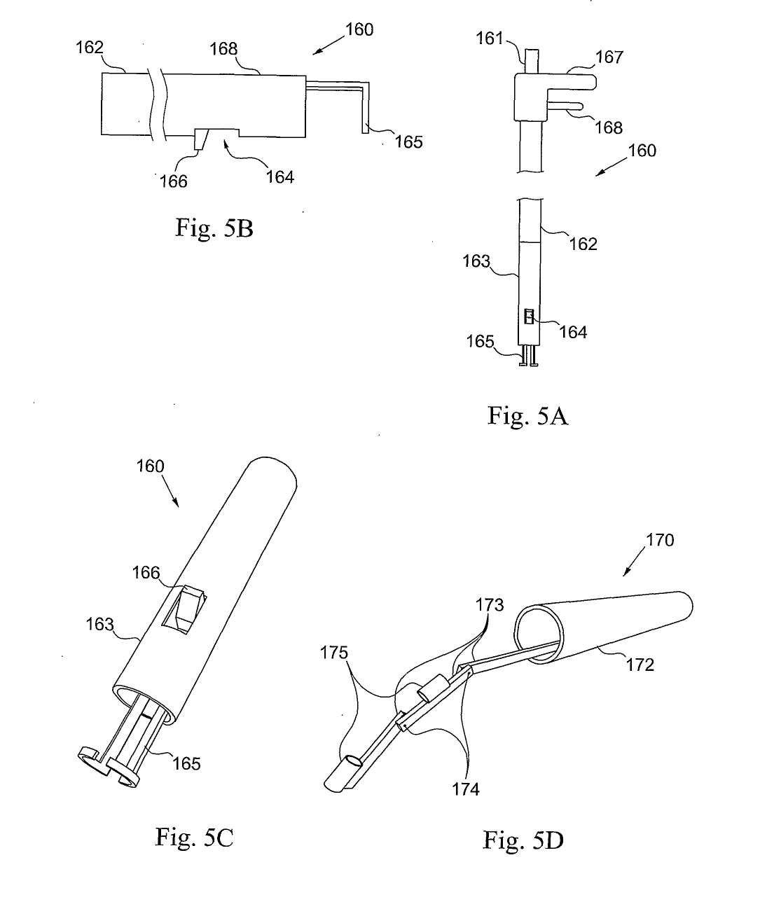

Releasing off a surgical effector, and/or attaching it onto a slender shaft whose distal end disposed within the lumen of the patient's body, as well as introducing a new or different surgical effector, are accomplished according to the present invention by means of an introducer. In FIG. 5A-5C elevational views and an isometric view of exemplary introducer 160 are respectively shown. (The same numbers indicate the same parts in FIGS. 5A-5C.) Shaft 161 is axially disposed within the lumen of slender pipe 162. Gripper 163 having aperture 164 and a pair of holding arms 165 is attached to the distal end of pipe 162. Arms 165 can be moved between two positions the first of which is a closed position in which both arms are retracted one towards the other thereby tightly gripping a surgical effector. In a second position the arms are fully extended thereby releasing a gripped effector. The arms are moved into the second position by inwardly pressing the proximal end of shaft 161 into pipe 162. A biasing spring, not shown presses against both arms to close back and simultaneously to outwardly push shaft 161 to its normal extended position whilst the proximal end of shaft 161 is being released. Hook 166 is extended off aperture 164 by puling handle 168 towards handle 166. A pair of inner cables, not shown, one of which connects hook 166 to handle 168 and the other connects hook 166 to another biasing spring, provides for extending and/or withdrawing hook 166 through aperture 164. A surgeon may release the above described locking mechanism of a surgical device by first extending hook 166, then engaging it with the locking mechanism and further pulling back hook 166.

An isometric view of introducer 170 of a system for telesurgery according to a preferred embodiment of the present invention is shown in FIG. 5D being at an extended position. External pipe 172 whose radius is relatively large, such as of 10 mm, encloses articulated inner shaft its distal segments 173 are pivotally attached to each other by hinges 174. Cylindrical receptacles 175 respectively attached to segments 173 provide for enclosing various surgical effectors to be introduced into the lumen of a patient's body enclosing the surgical site. Pairs of cables, not shown, wounded around pulleys mounted onto the proximal end of the slender shaft, such that one end of each cable of a pair is attached to the same respective segment of the inner shaft, whereas the other end is attached to a respective stretching/releasing mechanism, not shown, provide for inclining the respective segment of the inner shaft relative to the axis of pipe 172. Handles and operative levers provide for selecting a specific pair and either for respectively stretching and releasing each of its cables; and/or selecting all pairs and by similarly concomitantly stretching or releasing, to thereby extending and/or contracting the inner shaft off or into pipe 172, as known. The surgical effectors are attached to the inner surfaces of receptacles 175 by friction or optionally by a snatch locking mechanism as known.

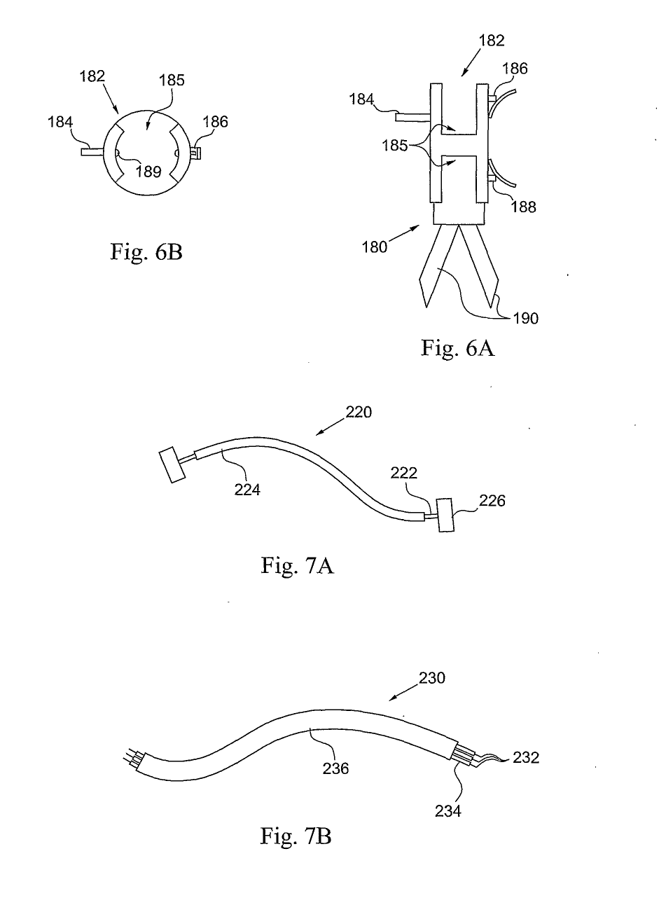

Elevational views of surgical effector 180 attached to mounting frame 182 each of which is in accordance with a preferred embodiment of the present invention are shown in FIGS. 6A and 6B respectively. (The same numbers indicate the same parts in FIGS. 6A-6B.) Gripping lever 184 laterally extends off a cylindrical wall of mounting frame 182. Both external and internal radii of cylindrical segments 185 respectively fit in with the radii of surgical effectors, such as effector 180, as well as with the radii of slender shafts, such as shaft 140 or 150 described above with reference to FIGS. 4A-4B to which reference is again made. Reference is now made to FIGS. 6A-8B. Locking devices 186 and 188 are of the type of snapping mechanism. Each of them consists of a lever having a spherical end, inwardly protruding from the inner side of the cylindrical wall opposing the wall to which gripping handle 184 is attached. Each such lever passes through a respective bore drilled through the cylindrical wall. The other end of the levers is resiliently attached to the opposite side of the wall, such as by a flat spring. Longitudinal bulges such as bulge 189 are disposed on the inner face of the cylindrical walls opposing the walls onto which the locking devices are attached. One end of each of the bulges is respectively disposed adjacent to the open end of both cylindrical segments 185. The ends of both bulges are spaced apart from the respective ends of the cylindrical walls. The bulges fit in with recesses respectively disposed on the surfaces of the surgical effectors as well as on the distal end of the slender shafts. The bulges and respective recesses provide keying for properly orienting the mounting frame relative to the slender shaft or effector 180 while attached to either of them. For attaching mounting frame 182 to the distal end of a slender shaft of the invention, the slender shaft is first slid into the respective cylindrical lumen of mounting frame 182 tightly gripped by an introducer. Such sliding may continue along the full depth of the respective cylindrical lumen only when the recess of the slender shaft faces the bulge of the mounting frame. Otherwise bulge 189 stops such sliding unless the slender shaft is oriented accordingly by rotating about its longitudinal axis. The spherical end of lock 186 is inwardly pushed into its respective bore by such axially sliding. This end snaps into its respective recess disposed on the surface of the slender shaft when facing it. The mounting frame is secured to the slender shaft, such that it is axially oriented in a predefined direction by means of the longitudinal bulge and locking device 186. Alternatively, surgical effector 180 as well as a slender shaft of an operative unit of the invention can be attached to a mounting frame by means of screwing mechanism, bayonet attachment, hook and loop attachment, magnetic attraction, or any other such attaching mechanisms as known.

The inner faces of both arms 190 of surgical effector 180 can be sharpened like scissors or adapted for gripping, such as jaws of tongs or tweezers. Arms 190 are movable between two positions one of which is a closed position and the other is an extended position. A biasing spring, not shown housed within the body of effector 180 provides for retaining arms 190 either at a closed position or at an extended position, when are not forced otherwise by means of a dedicated shaft or lever enclosed by the slender shaft of the invention. A surgeon may continuously move the arms between these two positions and/or change between them by means of operating handles mounted onto the slender shaft of the respective master unit. Such handles are connected to the inner shaft by means of a pair of cables similarly to the operating handles of an introducer for extending/withdrawing its hook as described hereinabove.

A surgical effector may have jaws adapted for grasping, dissecting, cutting, needle holding, coagulating tissue, clip application, or the jaws may comprise a cartridge of staples, etc. Alternatively, a surgical effector is a common device such as but not limited to: knife blades, scissors, dissectors, graspers, scalpels, hooks, hemostatics, clipers, clip removers, needle holders, retractors, suction element, a hook for grasping, tissue dissection, or cutting, or a cartridge of staples. Interchangeable surgical effectors releasably attached to slender shafts of an operative end units, can be according to the invention relatively robust, having diameters within a range of 4 to 10 mm, whereas the shafts diameters are within a range of 1 to 3 mm. Therefore a small surgical aperture of 1-3 mm cut through the wall of the patient's body provide for introducing such slender shafts into the lumen enclosing the surgical site. However a significantly larger surgical aperture of 5-10 mm provides for introducing relatively larger surgical effectors by means of suitable introducers. Obviously small sized openings cut through the wall of a patient's body are less painful and leave an imperceptible scar. Therefore a single relatively large surgical aperture may be used according to the invention to support a number of small sized surgical apertures for introducing slender shafts of the respective operative end units.

Elevational views of exemplary linking accessories for a system for telesurgery of the invention are shown in FIGS. 7A-7B. Hydraulic pipe 220 consists of pipe 222 enclosed within protective cover 224 having connectors, such as connector 226 disposed at its both ends providing for snap connecting. Cable accessory 230 consists of a number of cables 232 each of which is threaded trough an individual pipe, such as pipe 234 providing for its being stretched. The inner surfaces of the pipes are covered with a material having a low coefficient of friction such as Teflon for minimizing friction forces exerted on the cables while being translated within the enclosing pipes. The pipes are further surrounded with protective cover 236.

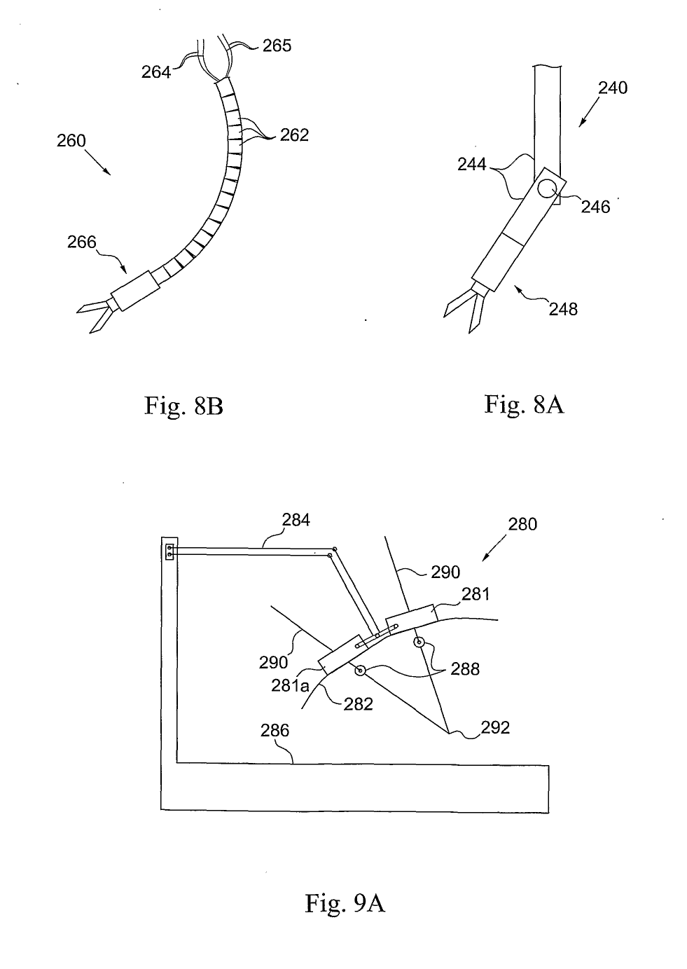

Two distal segments of slender shafts of an operative end unit of a system for telesurgery according to two other preferred embodiments of the present invention are schematically shown in FIGS. 8A-8B. The distal end of slender shaft 240 is articulated. Sub-segments 244 are pivotally attached to each other by means of hinges such as hinge 246. Surgical effector 248 is attached to the distal end of slender shaft 240. The angle between sub-segments 244 is changeable by means of inner cables respectively connected to each sub-segment at its both opposing sides. The distal segment of slender shaft 260 consists of a plurality of cylindrical rings 262. A pair of cables 264 respectively threaded through loops internally attached to the inner surface of each ring which are symmetrically disposed on the opposing inner faces of each ring provides for bending the distal segment. Cables 265 provide for manipulating the jaws of surgical effector 266. By means of such slender shaft an operator is able to access locations within the body of the patient which are covered by a tissue or an organ when viewed from the surgical aperture through which the slender shaft is introduced. Optionally both slender shafts can be such configured to provide for deflecting the surgical effectors in two different planes. Therefore a system for telesurgery having such slender shafts provides a surgeon with six degrees of freedom: three of them provide him for positioning the distal end of the slender shaft at a desired point in space; the fourth degree provides for axially rotating the slender shaft around its longitudinal axis, and the last two correspond to the two different planes in which the slender shaft can be deflected.

Viewing Subsystem

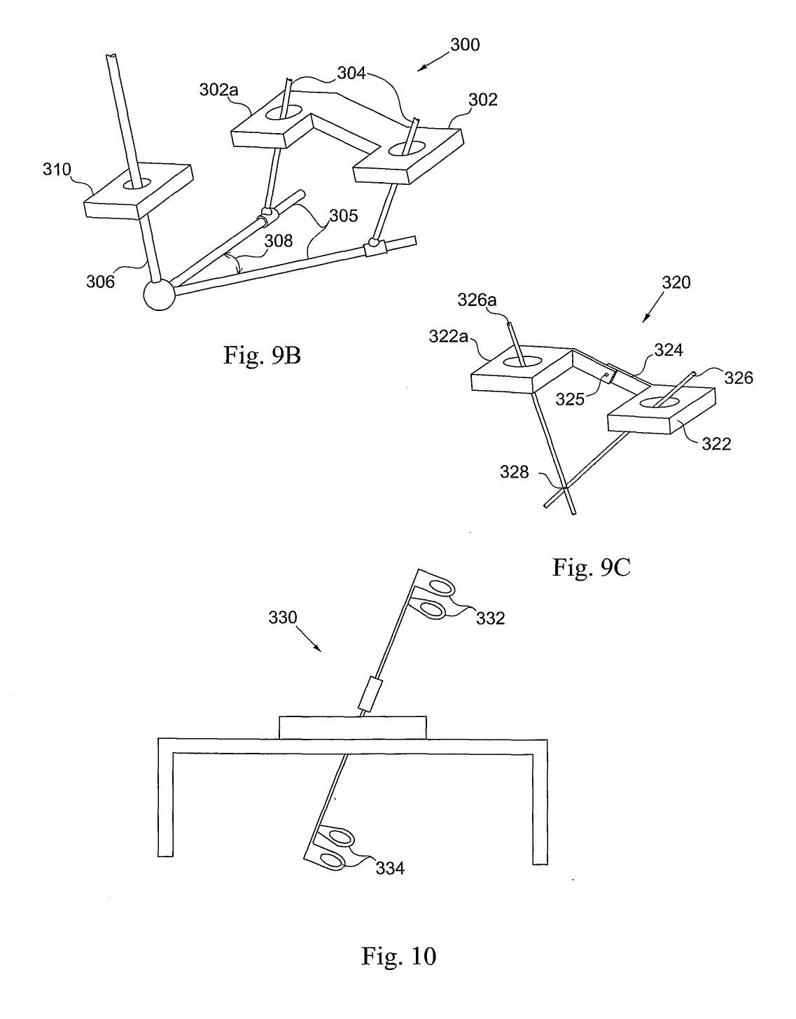

A viewing subsystem of a system for telesurgery of the invention includes a control member linked to a remote viewing member equipped with cameras and optional illuminating sources capable of being introduced into a lumen within the body of a patient. Reference is now made to FIGS. 9A-9B in which two members of a viewing subsystem of a system for telesurgery according to a preferred embodiment of the present invention are schematically shown respectively. Remote viewing member 280 has a pair of mutually connected operative end units 281 and 281a attached to surface 282 of a patient. Attaching is effected by means of articulated arm 284 connected to surgical bench 286 on which the patient is lying. Cameras 288 are respectively attached to the distal ends of slender shafts 290. Slender shafts 290 are introduced down to a pre-specified level beneath surface 282, and axially oriented such that both distal ends are spaced apart and separated by a predefined separation and the lines of sight of both cameras intersect at point 292 within the surgical site. The distance between both cameras 288 conforms to a normal distance between the human eyes.

Control member 300 has two mutually connected master units 302 and 302a oriented and spaced apart by the same orientation and separation as the operative end units of the respective remote viewing member are disposed relative to each other. The distal ends of slender shafts 304 are slidingly attached to two horizontal rods 305 each of which is pivotally attached to the distal end of slender shaft 306. The magnitude of angle 308 and the direction to which angle 308 points namely, the direction of its axis, as well as the angle between the plane containing angle 308 and the axis of the slender shaft 306, can be fixed and/or changed by the operator by means of operating handles of master end unit 310, not shown. Therefore the line of sight can be adjusted by the operator by respectively inclining, rotating and/or sliding slender shafts 304 following the adjusting of the magnitude and/or pointing of angle 308. Optionally slender shafts 304 and slender shaft 306 are simultaneously connected to an actuating device operated by a joystick as known, providing the operator for conveniently pointing both cameras such that the best field of view and focusing of the surgical site are received.

The control member of the viewing subsystem of a system for telesurgery in accordance with another preferred embodiment of the present invention consists of a pair of common modules, such as pair 320 schematically shown in FIG. 2C. The separation and relative orientation of base plates 322 and 322a can be manually fixed by means of brackets 324 and bolt 325. Slender shafts 326 and 326a are pivotally attached to each other by hinge 328 distally disposed relative to base plates 322 and 322a. Each common module of the pair is linked to its respective module of the viewing member as described hereinabove. Optionally to sliding bearing pivotally attached to each other substitute hinge 328 thereby enabling distally or proximally displacing the axis of rotations of one of the slender shafts relative to the other relative to baseplates 322 and 322a. The operator may push or pull and/or incline any of the slender shafts of the pair of common module of the control member and further adjust their rotational angle relative to their respective axes to thereby change the field of view and its center.

Both aforementioned control members further include binoculars, not shown, whose left and right eyepieces can be independently focused and are respectively connected to the left and right cameras of the remote viewing member. Links, not shown, including: a. mechanical transmissions for simultaneously sliding and/or axially rotating the respective slender shafts, and b. hydraulic transmissions providing for simultaneously inclining the respective slender shafts, of all master and operative end units of both the control and remote members, are further provided according to the present invention.

### Example

Reference is now made to FIG. 10 in which master unit 330 of a system for telesurgery according to a preferred embodiment is schematically shown. Operating handles such as handles 332 attached to the proximal end of the slender shaft and/or handles 334 attached to its distal end, provide for activating and manipulating the surgical effector of the respectively coupled operative end unit. Pushing and/or puling sideways handles 332, causes the slender shafts of master unit 330 and its respectively coupled operative end unit to be simultaneously inclined. Such inclination is to the same direction, by the same rotational angle, such that the proximal ends of both slender shafts move along the same vector describing the motion of the proximal end of the slender shaft of master unit 330. However the distal end of the respective surgical effector moves in the opposite direction by a proportional distance, whose magnitude depends on the ratio between the lengths of the segment of the slender shaft disposed distally to the center point and the segment proximally disposed to the same center point. However translating the distal end of the slender shaft of master unit 330 by moving handles 334 simultaneously moves the distal end of the surgical effector. Such movement is represented by the same vector describing the motion of the distal end of the shaft of master unit 330. Therefore the surgeon manipulating the surgical effector by means of handles 334 performs as in a normal open surgery, whereas manipulating by means of handles 332 is analogous to any normal endoscopic procedure. The system including a master unit such as master unit 330 may serve for any endoscopic operation within cavities, virtual cavities, artificially created cavities, including but not limited to: laparoscopy, retroperitoneoscopy, thoracoscopy, arthroscopy, percutaneous endoscopic surgery within cavities such as urinary bladder, stomach, heart, subcutaneous endoscopy (inguinal lymph node dissection; thyroid surgery, parathyroid surgery, breast augmentation and face lifting.

1. A system for telesurgery comprising
at least one operative end unit attachable to a surface of the body of a patient;
at least one master end unit for controlling and manipulating a member of said at least one operative end unit, and
wherein said at least one master unit and said at least one operative end unit each comprises a common module, and
wherein said common module comprises
a base plate having a distal surface;
a top member slidingly attached to said base plate;
a slender shaft having an axis, a proximal end and a distal end slidingly and pivotally attached to said base plate, wherein said proximal end proximally disposed relative to said base plate, and wherein said distal end distally disposed relative to said base plate, and
wherein any inclination of said slender shaft relative to said base plate simultaneously associated with a translation of said top member relative to said base plate, and wherein said at least one operative end unit linked to said at least one master unit, such that the slender shafts of both said at least one master end unit and said at least one operative end unit simultaneously slide each of which towards the same direction relative to its respective base plate.

2. A system for telesurgery as in claim 1, wherein both slender shafts of said at least one operative end unit and said at least one master end unit simultaneously slide by the same distance Δz.

3. A system for telesurgery as in claim 1, wherein said slender shaft is rotatable about a center point proximally disposed relative to said distal surface.

4. A system for telesurgery as in claim 3, wherein both slender shafts of said at least one operative end unit and said at least one master end unit simultaneously rotate about said center point by the same rotational angle.

5. A system for telesurgery as in claim 1, wherein said top member attached to said base plate by means of two sliding rods perpendicularly disposed one relative to the other, and wherein any rotation of said slender shaft about an axis parallel to one of said sliding rods simultaneously associated with a linear translation of the other sliding rod relative to said base plate.

6. A system for telesurgery as in claim 3, wherein said slender shaft is further axially rotatable.

7. A system for telesurgery as in claim 5, wherein both slender shafts of said at least one operative end unit and said at least one master end unit simultaneously rotated axially by the same rotational angle.

8. A system for telesurgery as in claim 1, wherein an operative handle is attached adjacent to the proximal end of at least one of said slender shafts.

9. A system for telesurgery as in claim 1, wherein an operative handle is attached adjacent to the distal end of at least one of said slender shafts.

10. A system for telesurgery as in claim 1, wherein a segment of at least one of said slender shafts is articulated.

11. A system for telesurgery as in claim 6, wherein a sub-segment of said segment is deflectable aside from said axis.

12. A system for telesurgery as in claim 1, wherein a surgical effector is attached to the distal end of at least one of said slender shafts, and wherein said surgical effector is any item selected from a group of items consisting of scissors, dissectors, graspers, scalpels, hooks, hemostatics, clipers, clip removers, needle holders, retractors and any combination thereof.

13. A system for telesurgery as in claim 12, wherein said surgical effector is interchangable.

14. A system for telesurgery as in claim 1, further comprising an introducer wherein said at introducer comprises at least one receptacle for enclosing a surgical effector.

15. A system for telesurgery as in claim 1, wherein said distal surface coated with an adhesive material for sealing a surgical aperture.

16. A system for telesurgery as in claim 1, wherein a transmission system provides for said linking, and wherein said transmission system comprises any item selected from a group of items consisting of cables, pulleys, hydraulic actuators, pneumatic actuators, electrical actuators, hydraulic pipes and any combination thereof.

17. A system for telesurgery as in claim 1, wherein a camera is attached to the slender shaft of said at least one operative end unit.

18. A system for telesurgery as in claim 1, wherein a light source is attached to the slender shaft of said at least one operative end unit.

19. A system for telesurgery as in claim 17, wherein said at least one operative end unit is the first operative end unit, and wherein a camera is attached to the distal end of the slender shaft of a second operative end unit, and wherein both said first and second operative end units spaced apart from each other, and wherein each of the first and second operative end units respectively linked to a first and second master end units similarly spaced apart from each other.

20. A system for telesurgery as in claim 19, wherein said cameras are spaced apart from each other by a pre-specified distance, and wherein said cameras are simultaneously movable such that each of said cameras points at the same point.
